(19) United States
(12) Patent Application Publication
(10) Pub. No.: US 2005/0116957 A1
(43) Pub. Date: Jun. 2, 2005

(54) Dynamic crop box determination for optimized display of a tube-like structure in endoscopic view ("crop box")

(75) Inventor: Yang Guang, Singapore (SG)

Correspondence Address: KRAMER LEVIN NAFTALIS & FRANKEL LLP, INTELLECTUAL PROPERTY DEPARTMENT, 919 THIRD AVENUE, NEW YORK, NY, 10022, US

(73) Assignee: Bracco Imaging, s.p.a., Milano (IT)

(21) Appl. No.: 10/981,109

(22) Filed: Nov. 3, 2004

Related U.S. Application Data
(60) Provisional application No. 60/517,043, filed on Nov. 3, 2003.
(60) Provisional application No. 60/516,998, filed on Nov. 3, 2003.
(60) Provisional application No. 60/562,100, filed on Apr. 14, 2004.

(51) Int. Cl. G06F 19/00; G01N 33/48; G01N 33/50; G06T 1/00

(52) U.S. Cl. 345/501; 702/19

(57) ABSTRACT

Methods and systems for dynamically determining a crop box to optimize the display of a subset of a 3D data set, such as, for example, an endoscopic view of a tube-like structure, are presented. In exemplary embodiments of the present invention, a “ray shooting” technique can be used to dynamically determine the size and location of a crop box. In such embodiments, shot rays are distributed evenly into a given volume and their intersection with the inner lumen determines the crop box boundaries. In alternate exemplary embodiments, rays need not be shot into fixed directions, but rather may be shot using a random offset which changes form frame to frame in order to more thoroughly cover a display area. In other exemplary embodiments, in order to get even better results, more rays can be shot at areas of possible error, such as, for example, where the centerline of a tube-like structure is leading to. In such embodiments rays need not be distributed evenly, but can be varied in space and time, i.e., In each frame the program can, for example, shoot out a different number of rays, in different directions, and the distribution of those rays could be in different pattern. Because, in exemplary embodiments, a dynamically optimized crop box encloses only the portion of the 3D data set which is actually displayed, processing cycles and memory usage are minimized.

### CROSS REFERENCE TO OTHER APPLICATIONS

This application claims the benefit of the following U.S. Provisional Patent Applications, the disclosure of each is hereby wholly incorporated herein by this reference: Ser. Nos. 60/517,043, and 60/516,998, each filed on Nov. 3, 2003, and Ser. No. 60/562,100, filed on Apr. 14, 2004.

### TECHNICAL FIELD

The present invention relates to the field of the interactive display of 3D data sets, and more particularly to dynamically determining a crop box to optimize the display of a tube-like structure in an endoscopic view.

### BACKGROUND OF THE INVENTION

Health care professionals and researchers are often interested in viewing the inside of a tube-like anatomical structure such as, for example, a blood vessel (e.g., the aorta) or a digestive system luminal structure (e.g., the colon) of a subject's body. Historically, the only method by which such users were able to view these structures was by insertion of an endoscopic probe and camera, as in a conventional colonoscopy or endoscopy. With the advent of sophisticated imaging technologies such as, for example, magnetic resonance imaging (“MRI”), echo planar Imaging (“EPI”), computerized tomography (“CT”) and the newer electrical impedance tomography (“EIT”), multiple images of various luminal organs can be acquired and 3D volumes constructed therefrom. These volumes can then be rendered to a radiologist or other diagnostician for a noninvasive inspection of the interior of a patient's tube-like organ.

In colonoscopy, for example, volumetric data sets can be compiled from a set of CT slices (generally in the range of 300-600, but can be 1000 or more) of the lower abdomen. These CT slices can be, for example, augmented by various interpolation methods to create a three dimensional volume which can be rendered using conventional volume rendering techniques. Using such techniques, a three-dimensional data set can be displayed on an appropriate display and a user can take a virtual tour of a patient's colon, thus dispensing with the need to insert an endoscope. Such a procedure is known as a “virtual colonoscopy,” and has recently become available to patients.

Notwithstanding its obvious advantages of non-invasiveness, there are certain inconveniences and difficulties inherent in virtual colonoscopy. More generally, these problems emerge in the virtual examination of any tube-like anatomical structure using conventional techniques.

For example, conventional virtual colonoscopy places a user's viewpoint inside the colon lumen and moves this viewpoint through the interior, generally along a calculated centerline. In such displays, depth cues are generally lacking, given the standard monoscopic display. As a result, important properties of the colon can go unseen and problem areas can remain unnoticed.

Additionally, typical displays of tube-like anatomical structures in endoscopic view only show part of the structure on the display screen. Generally, endoscopic views correspond only to a small portion of the entire tube-like structure, such as, for example, in terms of volume of the scan, from 2% to 10%, and in terms of length of the tube-like structure, from 5% to 10% or more. In displaying such an endoscopic view, if a display system renders the entire colon to display only a fraction of it, such a technique is both time consuming and inefficient. If the system could determine and then render only the portion to be actually displayed to a user or viewer, a substantial amount of processing time and memory space could thus be saved.

Further, as is known in the art of volume rendering, the more voxels that must be rendered and displayed, the higher the demand on computing resources. The demand on computing resources is also proportional to the level of detail a given user chooses, such as, for example, by increasing digital zoom or by increasing rendering quality. If greater detail is chosen, a greater number of polygons must be created in sampling the volume. When more polygons are to be sampled, more pixels are required to be drawn (and, in general, each pixel on the screen would be repeatedly filled many times), and the fill rate will be decreased. At high levels of detail such a large amount of input data can slow down the rendering speed of the viewed volume segment and can thus require a user to wait for the displayed image to fill after, for example, moving the viewpoint to a new location.

On the other hand, greater detail is generally desired, and is in fact often necessary, to assist a user in making a close diagnosis or analysis. Additionally, if depth cues are desired, such as, for example, by rendering a volume of interest stereoscopically, the number of sampled polygons that must be input to rendering algorithms doubles, and thus so does the memory required to do the rendering.

More generally, the above-described problems of the prior art are common to all situations where a user interactively views a large 3D data set one portion at a time, where the portion viewed at any one time is a small fraction of the entire data set, but where the said portion cannot be determined a priori. Unless somehow remediated, such interactive viewing is prone to useless processing of voxels which are never actually displayed, diverting needed computing resources from processing and rendering those voxels that are being displayed, introducing, among other difficulties, wait states.

Thus, what is needed in the art are optimizations to the process of displaying large 3D data sets where at many given moments the portion of the volume being inspected is only a subset of the entire volume. Such optimizations should more efficiently utilize computing resources and thus facilitate seamless no-wait state viewing with depth cues, greater detail and the free use of tools and functionalities at high resolutions that require large numbers of calculations for each voxel to be rendered.

### SUMMARY OF THE INVENTION

Methods and systems for dynamically determining a crop box to optimize the display of a subset of a 3D data set, such as, for example, a virtual endoscopic view of a tube-like structure, are presented. In exemplary embodiments of the present invention, a “ray shooting” technique can be used to dynamically determine the size and location of a crop box. In such embodiments, rays can be, for example, shot into a given volume and their intersection with the inner lumen can, for example, determine crop box boundaries. In exemplary embodiments of the present invention, rays need not be shot into fixed directions, but rather can be, for example, shot using a random offset which changes from frame to frame in order to more thoroughly cover a display area. In other exemplary embodiments, more rays can be shot at areas of possible error, such as, for example, in or near the direction of the furthest extent of a centerline of a tube-like structure from a current viewpoint. In such exemplary embodiments rays can be varied in space and time, where, for example, in each frame an exemplary program can, for example, shoot out a different number of rays, in different directions, and the distribution of those rays can be in different pattern. Because a dynamically optimized crop box encloses only the portion of the 3D data set which is actually displayed at any point in time, processing cycles and memory usage used in rendering the data set can be significantly minimized.

Further features of the invention, its nature and various advantages will be more apparent from the accompanying drawings and the following detailed description of the various exemplary embodiments.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates an exemplary virtual endoscopic view of a portion of a human colon;

FIG. 1(a) is a greyscale version of FIG. 1.;

FIG. 2 illustrates an exemplary current view box displayed as a fraction of an entire structure view of an exemplary human colon;

FIG. 2(a) is a greyscale version of FIG. 2;

FIG. 3 depicts exemplary rays shot into a current virtual endoscopic view according to an exemplary embodiment of the present invention;

FIG. 3(a) is a greyscale version of FIG. 3;

FIG. 4 depicts a side view of the shot rays of FIG. 3;

FIG. 4(a) is a greyscale version of FIG. 4;

FIG. 5 illustrates an exemplary crop box defined so as to enclose all hit points from rays shot according to an exemplary embodiment of the present invention;

FIG. 6 depicts an exemplary set of evenly distributed ray hit points used to define a crop box where a farthest portion of the colon is not rendered according to an exemplary embodiment of the present invention;

FIG. 6(a) is a greyscale version of FIG. 6;

FIG. 7 depicts the exemplary set of hit points of FIG. 6 augmented by an additional set of hit points evenly distributed about the end of the depicted centerline, according to an exemplary embodiment of the present invention;

FIG. 7(a) is a greyscale version of FIG. 7;

FIGS. 8(a)-(d) depict generation of a volume axes aligned crop box and a viewing frustrum aligned crop box according to various embodiments of the present invention.

FIGS. 9(a) and (b) illustrate an exemplary large sampling distance (and small corresponding number of polygons) used to render a volume;

FIGS. 9(c) and (d) are greyscale versions of FIGS. 9(a) and (b), respectively;

FIGS. 10(a) and (b) illustrate, relative to FIG. 9, a smaller sampling distance (and larger corresponding number of polygons) used to render a volume;

FIGS. 10(c) and (d) are greyscale versions of FIGS. 10(a) and (b), respectively;

FIGS. 11(a) and (b) illustrate, relative to FIGS. 10, a still smaller sampling distance (and still larger corresponding number of polygons) used to render a volume;

FIGS. 11(c) and (d) are greyscale versions of FIGS. 11 (a) and (b), respectively;

FIGS. 12(a) and (b) illustrate, relative to FIG. 11, a still smaller sampling distance (and still larger corresponding number of polygons) used to render a volume;

FIGS. 12(c) and (d) are greyscale versions of FIGS. 12(a) and (b), respectively;

FIGS. 13(a) and (b) illustrate an exemplary smallest sampling distance (and largest corresponding number of polygons) used to render a volume;

FIGS. 13(c) and (d) are greyscale versions of FIGS. 13(a) and (b), respectively;

FIG. 14 depicts shooting rays with a random offset according to an exemplary embodiment of the present invention; and

FIG. 14(a) is a greyscale version of FIG. 14.

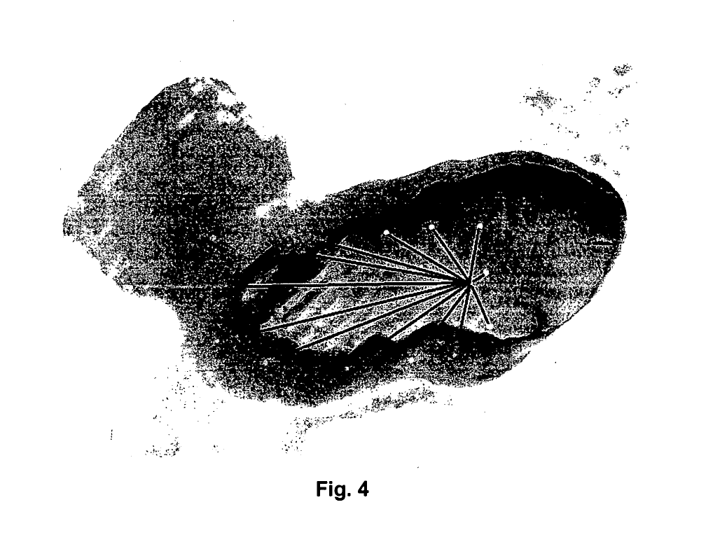

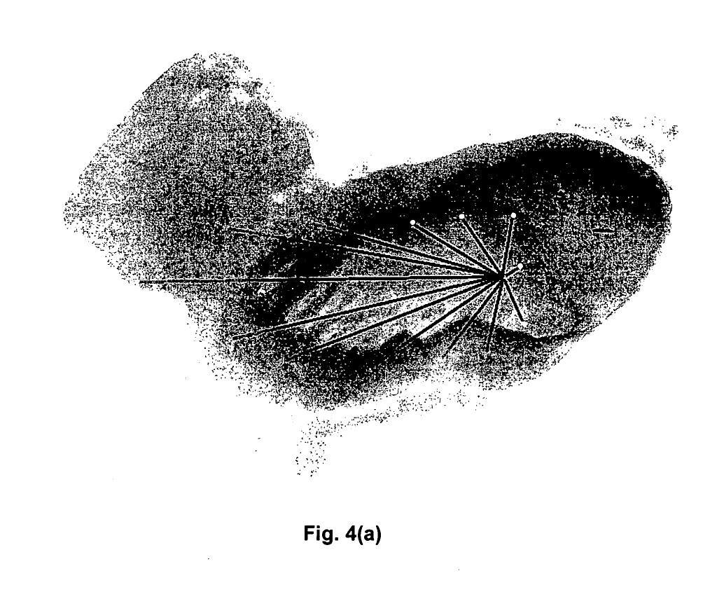

It is noted that the patent or application file contains at least one drawing executed in color. Copies of this patent or patent application publication with color drawings will be provided by the U.S. Patent Office upon request and payment of the necessary fee. For illustrative purposes grayscale drawings are also provided of each color drawing. In the following description the color and grayscale version of a given figure will be collectively referred to as that figure (e.g., “FIG. 4” includes “FIG. 4” and “FIG. 4(a)”, its grayscale component), it being understood that all versions of the figure are included.

### DETAILED DESCRIPTION OF THE INVENTION

Exemplary embodiments of the present invention are directed towards using ray-shooting techniques to increase the final rendering speed of a viewed portion of a volume.

In rendering a volume, a final rendering speed is inversely proportional to the following factors: (a) input data size—the larger the data size, the more memory and CPU time consumed in rendering it; (b) physical size of texture memory of the graphic card, vs. the texture memory the program requires—if the texture memory required exceeds the physical texture memory size, texture memory swapping will be involved, which is an expensive operation. In practice this swap can happen frequently when processing a large amount of data, thus resulting in a drastic decrease in performance; (c) size of volume to be rendered, at current moment (crop box)—the smaller the crop box is, the lesser the number of polygons that are needed to be sampled and rendered; (d) detail of the rendering (i.e., the number of polygons used)—the higher the detail, the more polygons are needed; and (e) use of shading—if shading is enabled, four times the texture memory is required.

Thus, if one or more of the above factors can be optimized, the final rendering speed will be increased. In exemplary embodiments of the present invention, this can be achieved by optimizing the size of a crop box.

In exemplary embodiments of the present invention, a crop-box's size can be calculated using a ray-shooting algorithm. In order to apply such an exemplary algorithm efficiently, the following issues need to be addressed:

a. Number of rays shot per display frame. Theoretically, the more the better, but the more rays shot, the slower the processing speed is;

b. Distribution of the rays in the 3D space. The rays should cover all of the surface of interest. To achieve this, in exemplary embodiments of the present invention, the arrangement of the rays can be, for example, randomized, so greater coverage can be obtained for the same number of rays. For areas needing more attention, more rays can, for example, be shot toward them; for areas that need less attention, a lesser number of rays can be used; and

c. Use of the ray shooting result (single frame v. multiple frames). In exemplary embodiments of the present invention, in each frame the hit-points results can be collected. In one exemplary implementation this result can be used locally, i.e., in the current display frame, and discarded after the crop box calculation; alternatively, for example, the information can be saved and used for a given number of subsequent frames, so a better result can be obtained without having to perform additional calculations.

The present invention, for illustration purposes, will be described using an exemplary tube-like structure such as, for example, a colon. The extension to any 3D data set where only a small portion of same is visualized by a user at any one time, is fully contemplated within the scope of the invention.

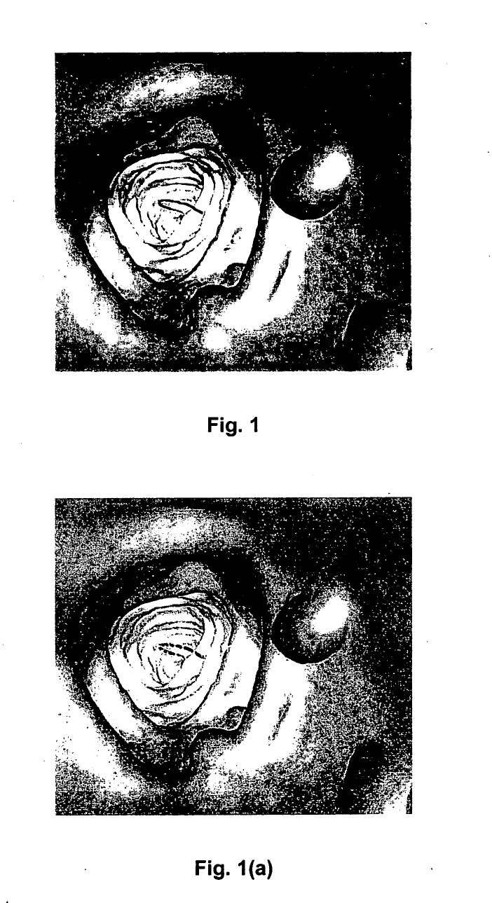

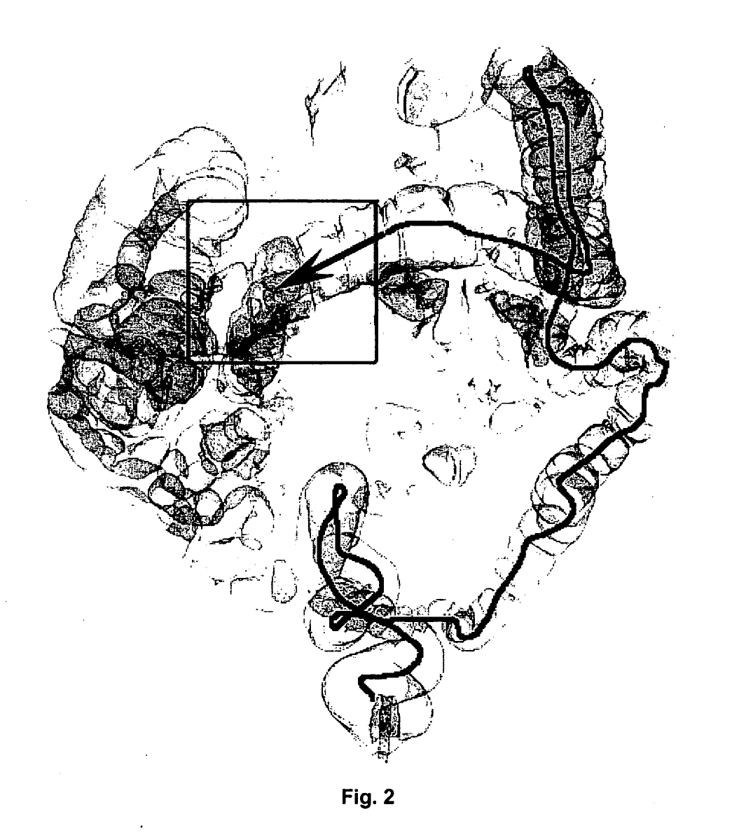

In exemplary embodiments according to the present invention, a 3D display system can determine a visible region of a given tube-like anatomical structure around a user's viewpoint as a region of interest, with the remaining portion of the tube-like structure not needing to be rendered. For example, a user virtually viewing a colon in a virtual colonoscopy generally does not look at the entire inner wall of the colon lumen at the same time. Rather, a user only views a small portion or segment of the inner colon at a time. FIG. 1 illustrates such an exemplary endoscopic view of a small segment of the inner colon. Such a segment can be selected for display, for example, as illustrated in FIG. 2, by forming a box around an area of interest within the whole structure. The selected segment generally fills the main viewing window, as shown in FIG. 1, so that it can be seen in adequate detail. Thus, as a user's viewpoint moves through the colon lumen, it is not necessary to render the entire volumetric data set containing the entire colon, but rather only the portion that the user will see at any given point in time. Not having to render voxels that are invisible to a user from his then current viewpoint greatly optimizes system performance and decreases the load on computing resources. In exemplary embodiments of the present invention, the load can be decreased to be only 3% to 10% of the whole scan, a significant optimization.

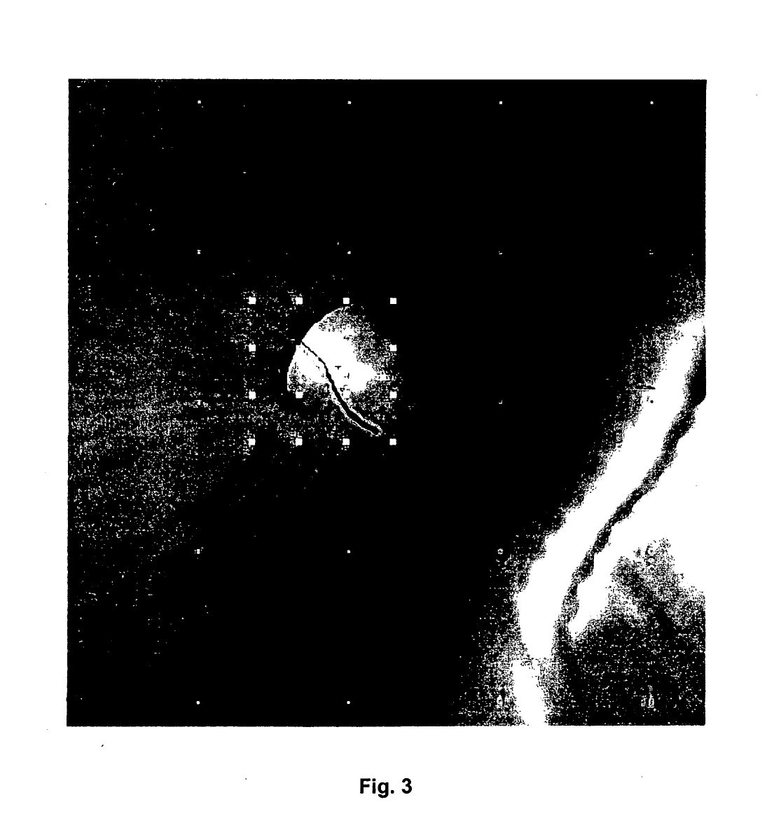

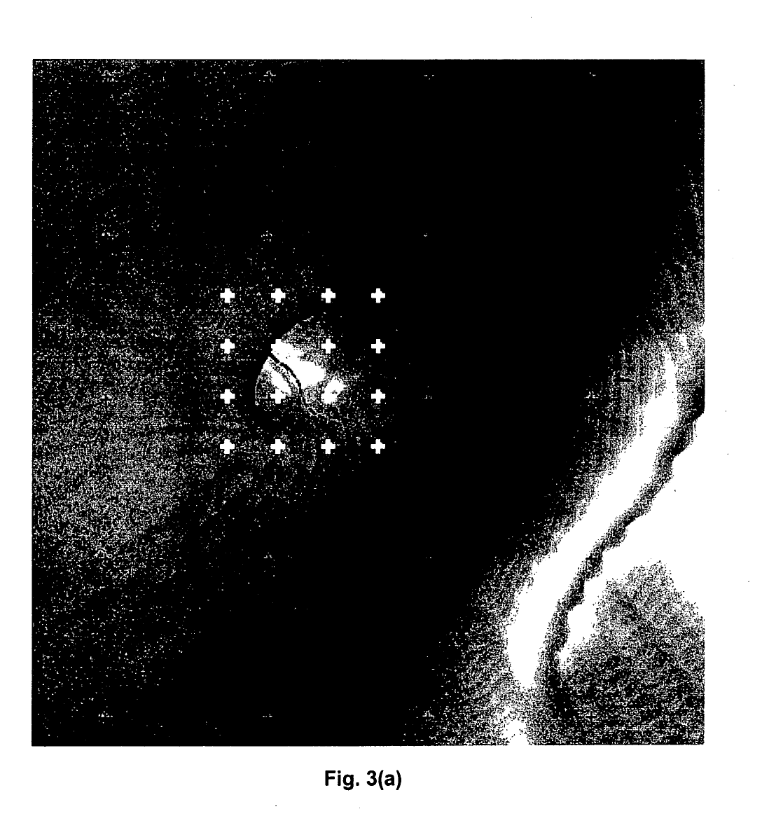

Thus, in exemplary embodiments according to the present invention, a “shooting ray” method can be used. For example, a ray can be constructed starting at any position in the 3D model space and ending at any other position in the 3D model space. Such “ray shooting” is illustrated in FIGS. 3 and 4, where FIG. 3 illustrates shooting rays into a current endoscopic view of a colon and FIG. 4 shows the shooting rays as viewed from the side. By checking the values of each voxel that the ray passes through relative to a defined threshold value, such an exemplary system can obtain information regarding the “visibility” of any two points. Voxels representing the air between lumen walls are “invisible”, and a ray can pass through them. Upon reaching the first “visible” voxel, the location of a voxel on the inner lumen wall has been acquired. Such a location will sometimes be referred to as a “hit point.”

In exemplary embodiments of the present invention, an algorithm for such ray shooting can be implemented according to the following exemplary pseudocode.

A. Pseudo Code For Distribute_Rays:
In every rendering loop:
Determine the projection width and height; // (if varying—if not see below)
Divide the projection plane into m by n grids, each grid having size (width/m) by (height/n); and
Shoot one ray from the current viewpoint towards the center of each grid.

The integers m and n can, for example, be both equal to 5, or can take on such other values as are appropriate in a given implementation. In many exemplary embodiments the projection width and height is a known factor, such as for example, in any OpenGL program (where it is specified by the user), and thus it does not always change; thus, there is no need to determine these values in every loop in such cases.

B. Pseudo Code For Ray_Shooting:
For each ray,
1. from the starting point of the ray towards the direction of this ray, pick up the first voxel along the path,;
2. for the voxel, check if the voxel's intensity value excess a certain threshold;
if yes,
3. then it's a “solid” voxel and take it's position as the “hit point's” position, return;
if no,
4. go to pick up the next voxel along the path, go to 2;
5. if there is no voxel to pick up (e.g., the ray goes out of the volume), return;

In exemplary embodiments of the present invention the direction of the ray is simply that from the current viewpoint to the center of each grid, and can be, for example, set as follows:
ray.SetStartingPoint(currentViewpoint.GetPosition( ));
ray.SetDirection(centerOfGrid—currentViewpoint.GetPosition( ));

C. Pseudo Code For Calculating_Bounding_Box:
for all the coordinates (x, y, z) of “hit points”, find the minimum and maximum values as Xmin, Xmax, Ymin, Ymax, Zmin, and Zmax, respectively. The box with one corner (Xmin, Ymin, Zmin) and the opposite corner (Xmax, Ymax, Zmax) is the bounding box needed.

Thus, by using such a “shooting ray” method, in exemplary embodiments of the present invention a system can, for example, construct an arbitrary number of rays from a user's current viewpoint and send them in any direction. Some of these rays (if not all) will eventually hit a voxel on the inner lumen wall along their given direction; this creates a set of “hit points.” The set of such hit points thus traces the extent of the region that is visible from that particular viewpoint. In FIGS. 3 and 4, for example, the resultant hit points are shown as either yellow or cyan colored dots in color drawings, or white crosses and black crosses in grayscale drawings, respectively. The cyan dots (black crosses) shown in FIG. 3 illustrate, for example, the hit points generated by a group of rays evenly distributed into the visible area. The yellow dots (white crosses) indicate the hit points for another set of shot rays that were targeted to only one portion of the volume, centered at the end of the centerline of an exemplary colon lumen. Since each of the distances from a hit point to a user's viewpoint can be calculated one by one, this technique can be used to dynamically delineate a visibility box from any given viewpoint. The voxels within such a visibility box are thus the only voxels that need to be rendered when the user is at that given viewpoint. A visibility box can, for example, have an irregular shape. For ease of computing, a exemplary system can, for example, enclose a visibility box by a simply shaped “crop box,” being, for example, a cylinder, sphere, cube, rectangular prism or other simple 3D shape.

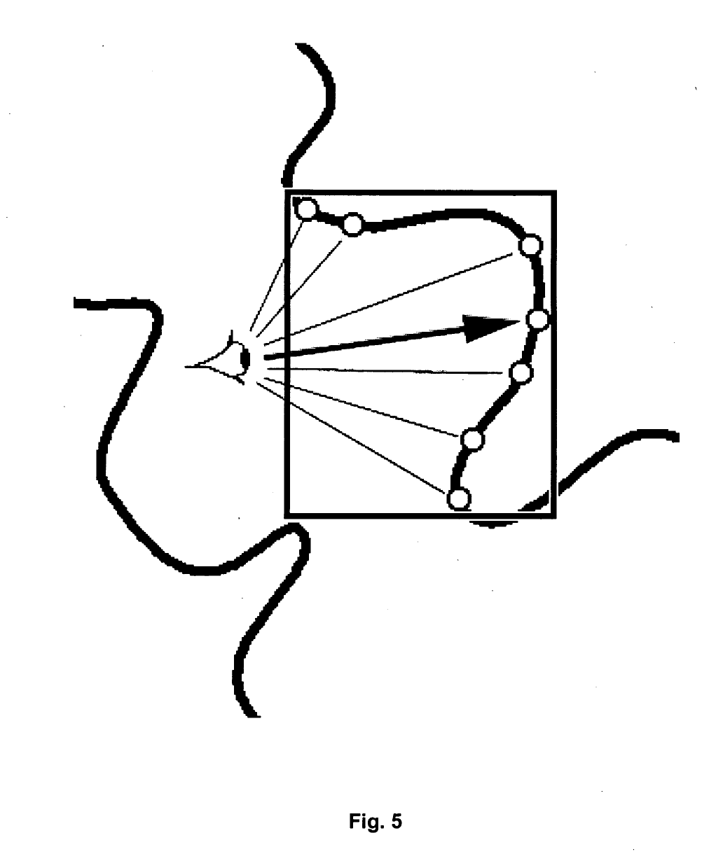

The above described method is further illustrated in FIG. 5. With reference thereto, a user's viewpoint is indicated in FIG. 5 by an eye icon. From this viewpoint, exemplary rays can be, for example, shot in a variety of directions which hit the surface of the structure at the shown points. A rectangular region can then be fitted so as to contain all of the hit points within a certain user-defined safety margin.

In exemplary embodiments of the present invention a bounding box can be generated, for example, with such a defined safety margin, as follows:

D. Pseudocode For Calculate_Bounding_Box_Saftey_Margin:
For a bounding box with corners (Xmin, Ymin, Zmin) and (Xmax, Ymax, Zmax), pad an offset to it so that the box becomes (Xmin-offset, Ymin-offset, Zmin-offset) and (Xmax-offset, Ymax-offset, Zmax-offset);
where offset can be the same, or can be separately set for each of the X, Y and Z directions.

Such a rectangular region, in exemplary embodiments of the present invention, can, for example, encompass a visibility region with reference to the right wall of the tube-like structure, as depicted in FIG. 5. A similar technique can be, for example, applied to the left wall, and an overall total crop box thus created for that viewpoint.

In exemplary embodiments according to the present invention, it can be common that, for example, 40 to 50 such rays, spread throughout a user's current field of view, can be able to collect sufficient information regarding the geography of the tube-like structure's surface so as to form a visibility region. In exemplary embodiments of the present invention, the number of rays that is shot is adjustable. Thus, the more rays that are shot the better the result, but the slower the computation. Thus, in exemplary embodiments of the present invention the number of rays shot can be an appropriate value in given contexts which balances these two factors, i.e., computing speed and required accuracy for crop box optimization.

In the above described pseudo code for calculate_bounding_box, where the following function is stated {for all the coordinates (x, y, z) of “hit points”}, if the “hit points” are not only from the current frame, but also from the previous several frames, in exemplary embodiments of the present invention, a bounding box can still be accurately calculated. In fact, if enough information from previous frames is saved, the result can, in exemplary embodiments, be even better.

In exemplary embodiments of the present invention, hit points from previous frames can be utilized as follows:

E. Pseudocode For Using Previous Hit Points In Subsequent Frames:

For each display loop,
hit points=ShootRays; //as above
hit_points_pool.add(hit_points); //add new hit points into a “pool”,
   //a storage
determine the crop box using all the hit points in hit_points_pool;
   //previously was using just the current hit_points,
   //previous loops' hit_points has been deleted,
   //and never re-used

In exemplary embodiments of the present invention a hit_points_pool can, for example, store the hit_points from both the current as well as previous (either one or several) loops. Thus, in each loop the number of hit_points used to determine the crop box can be greater than the number of rays actually shot out; thus, all hit_points can be, for example, stored into a hit_points_pool and re-used in following loops.

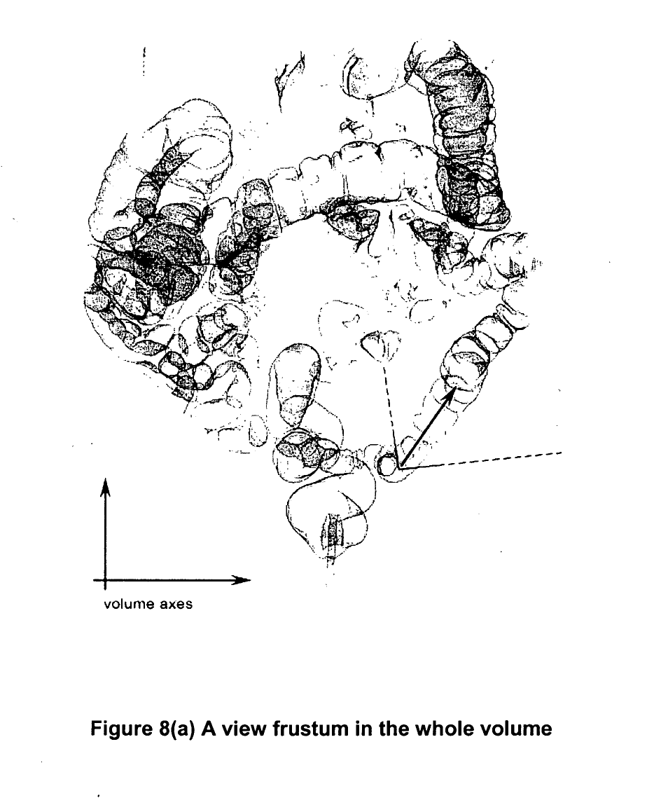

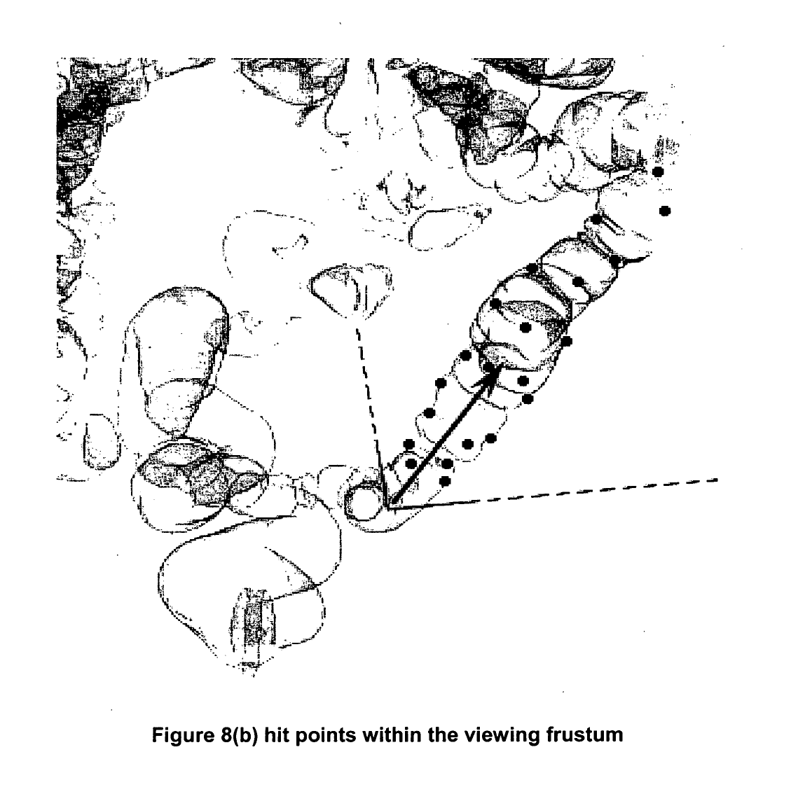

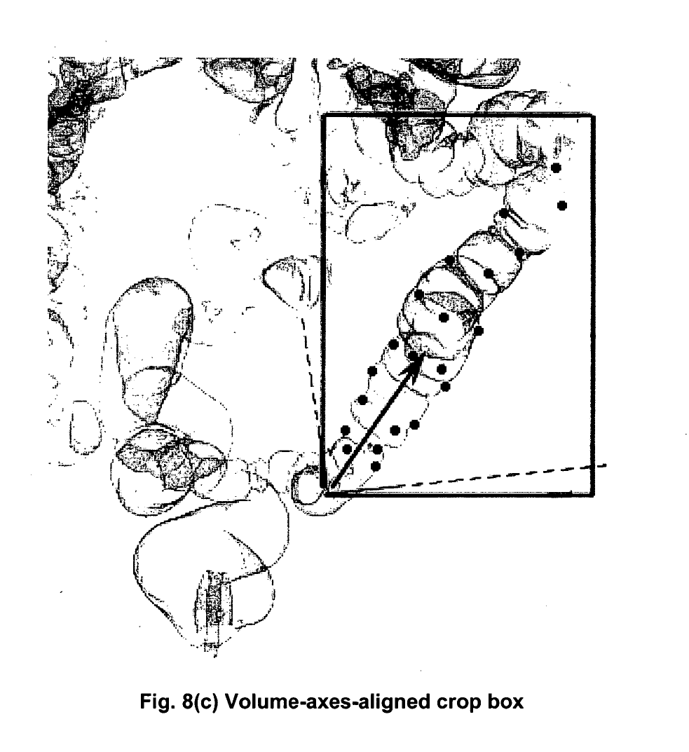

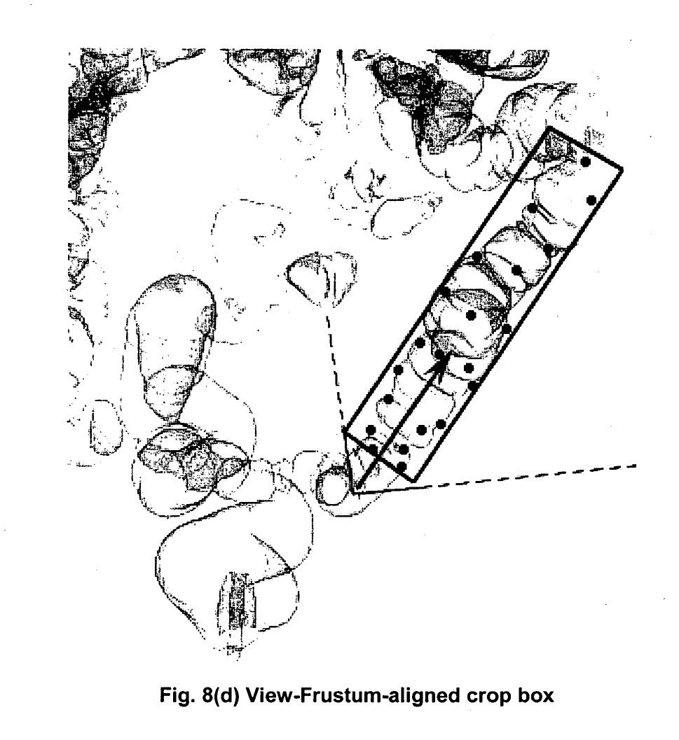

As noted above, by collecting information regarding the hit points, in exemplary embodiments of the present invention, the coordinates of such hit points can be utilized to create an (axis-aligned) crop box enclosing all of them. This can define a region visible to a user, or a region of interest, at a given viewpoint. Such a crop box can be used, for example, to reduce the actual amount of the overall volume that needs to be rendered at any given time, as described above. It is noted that for many 3D data sets an ideal crop box may not be axis-aligned (i.e., aligned with the volume's x, y and z axes), but can be, for example, aligned with the viewing frustrum at the given viewpoint. Such alignment further decreases the size of a crop box, but can be computationally more complex for the rendering. FIGS. 8(a)-(d) depict the differences between an axis aligned crop box and one that is viewing frustrum aligned. Thus, in exemplary embodiments of the present invention where it is feasible and desirable to free-align the crop box, the crop box can be, for example, viewing frustum aligned, or aligned in any other manner which is appropriate given the data set and the computing resources available.

Such an exemplary free-aligned crop box is illustrated with reference to FIGS. 8. FIG. 8(a) depicts an exemplary viewing frustrum at a given viewpoint in relation to an entire exemplary colon volume. As can be seen, there is no particular natural alignment of such a frustrum with the axes of the volume. FIG. 8(b) depicts exemplary hit points, obtained as described above. FIG. 8(c) depicts an exemplary volume-axes aligned crop box containing these hit points. As can be seen, the crop box has extra space in which no useful data appears. Nonetheless, these voxels will be rendered in the display loop. FIG. 8(d) depicts an exemplary viewing frustrum-aligned crop box, where the crop box is aligned to the viewpoint direction and directions orthogonal to that direction vector in 3D space. As can be seen, such a crop box “naturally” fits the shape of the data, and can thus be significantly smaller, however, in order to specify the voxels contained within it an exemplary system may need, in exemplary embodiments of the present invention, to implement co-ordinate transformation, which can be computationally intense.

In exemplary embodiments, the size of a crop box can be significantly smaller than the volume of the entire structure under analysis. For example, in exemplary embodiments of the present invention, it can be 5% or less of the original volume for colonoscopy applications. Accordingly, rendering speed can be drastically improved.

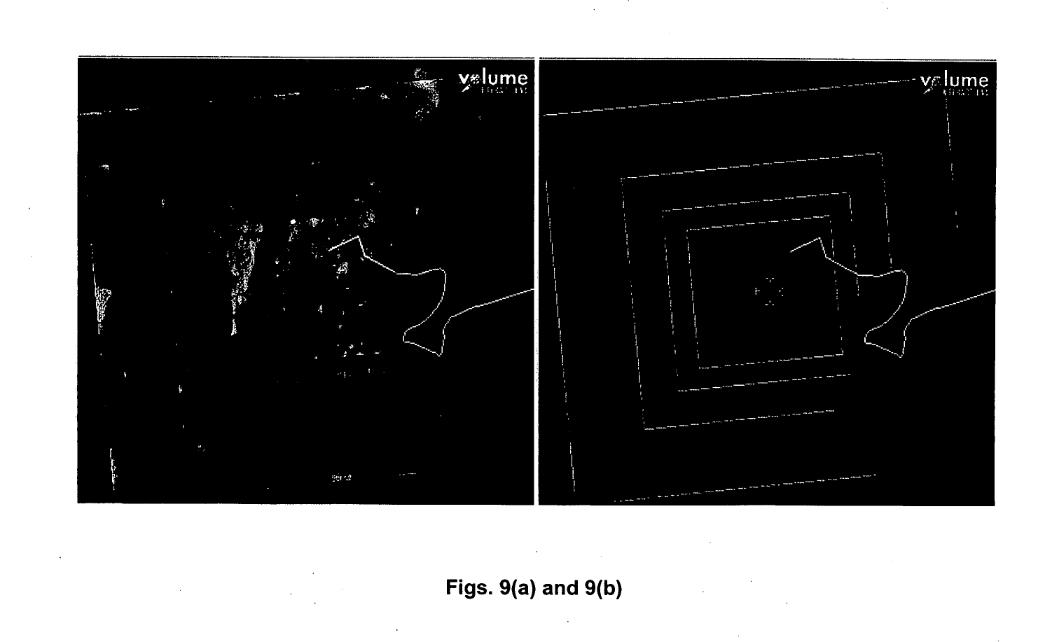

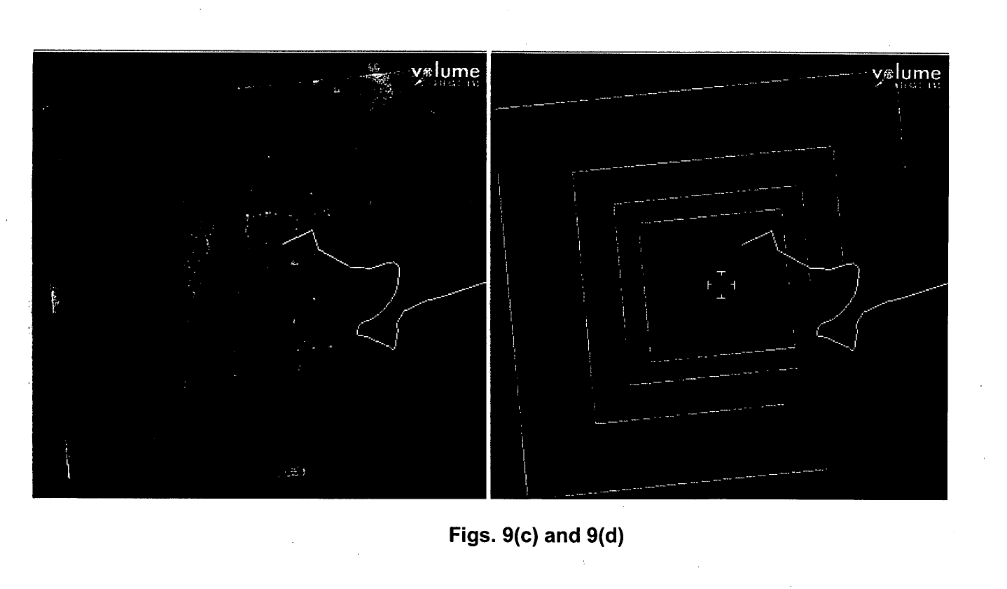

As noted above, rendering speed depends upon many factors. FIGS. 9-13 illustrate the relationship between sampling distances (i.e., the distances between polygons perpendicular to the viewing direction used to resample the volume for rendering), number of polygons required to be drawn, rendering quality, and crop box.

The left parts of each of FIGS. 9-13 (i.e., the portions of the figures denoted (a) and (c)) show the textured polygons, and the right parts (i.e., those portions of the figures denoted (b) and (d)) show only the edges of the polygons. At any given moment, the dimensions of all the polygons shown actually form a cuboid shape, which reflects the fact that the sizes of the polygons are determined by the crop box, which is calculated prior to this stage, i.e., the crop box is calculated immediately prior to displaying, in every display loop. So, in fact, the polygons indicate the shape of the crop box.

FIG. 9 was created by purposely specifying a very large sampling distance, which results in very few polygons used in resampling. This gives very low detail. The number of polygons shown in FIG. 9 is only about 4 or 5.

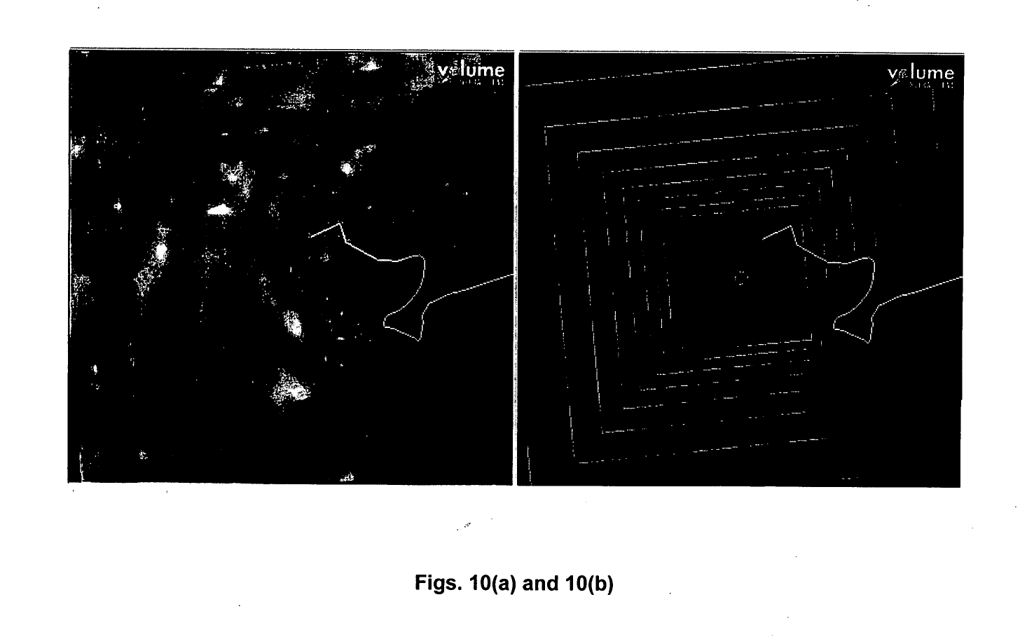

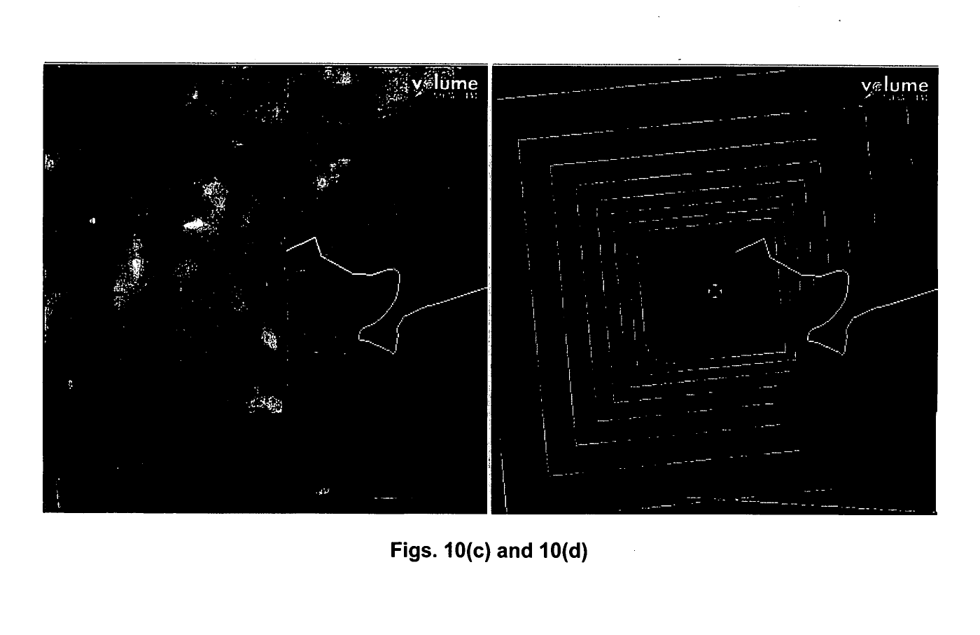

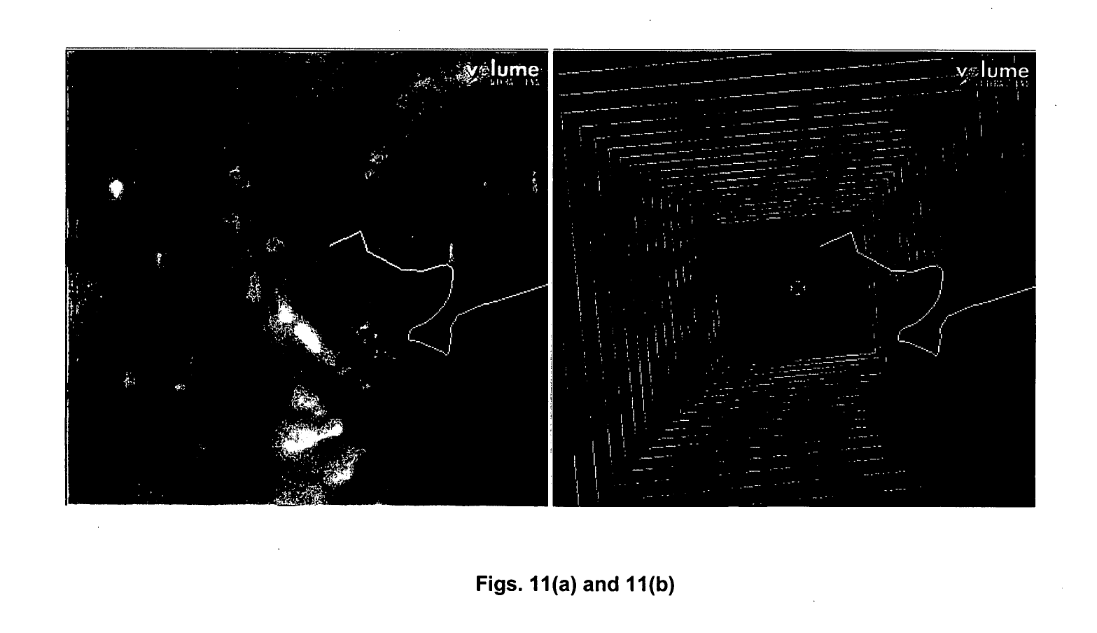

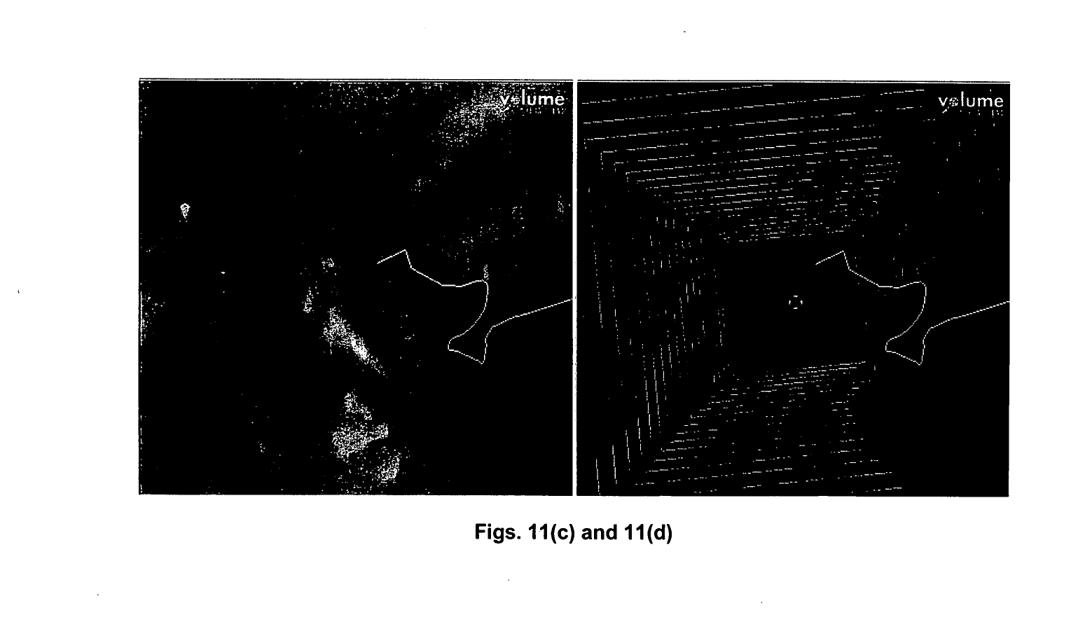

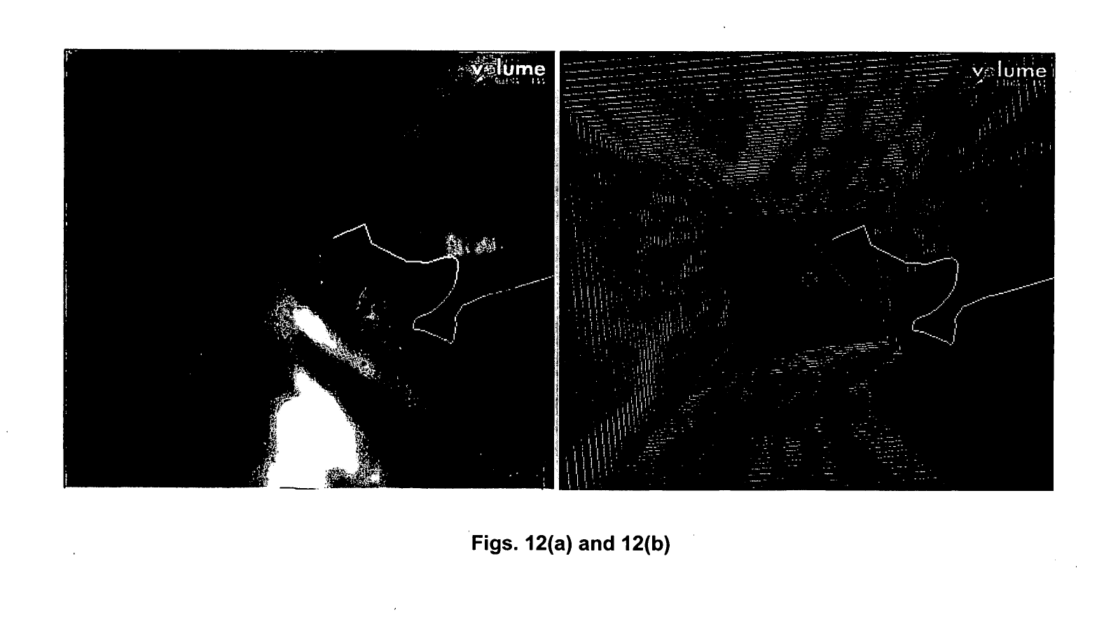

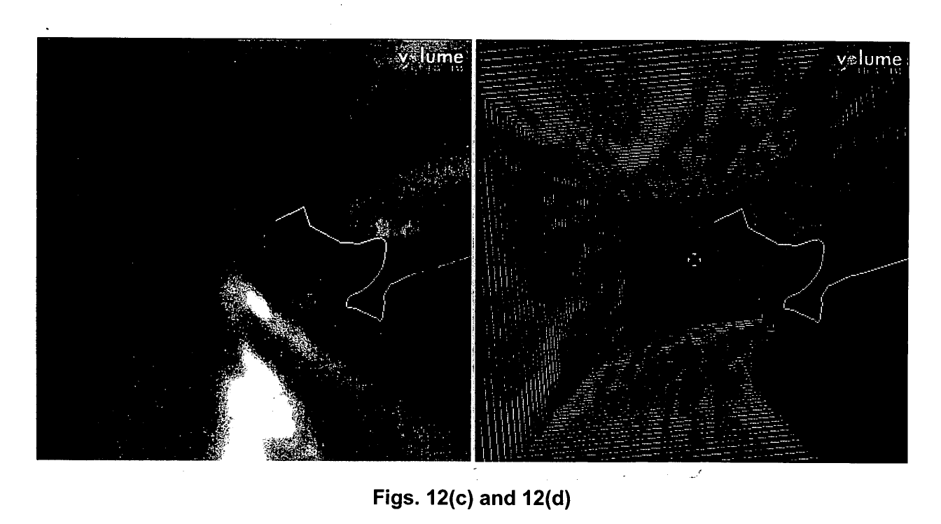

In FIG. 10 the sampling distance has been decreased, therefore the amount of polygons are increased. At this value the image is still meaningless, however. FIGS. 11 and 12 depict the effect of a further decrease in the sampling distance (and corresponding increase in sampling distance) and thus give more detail, and the shape of the lumen appears to be more recognizable as a result. The number of polygons has increased drastically, however.

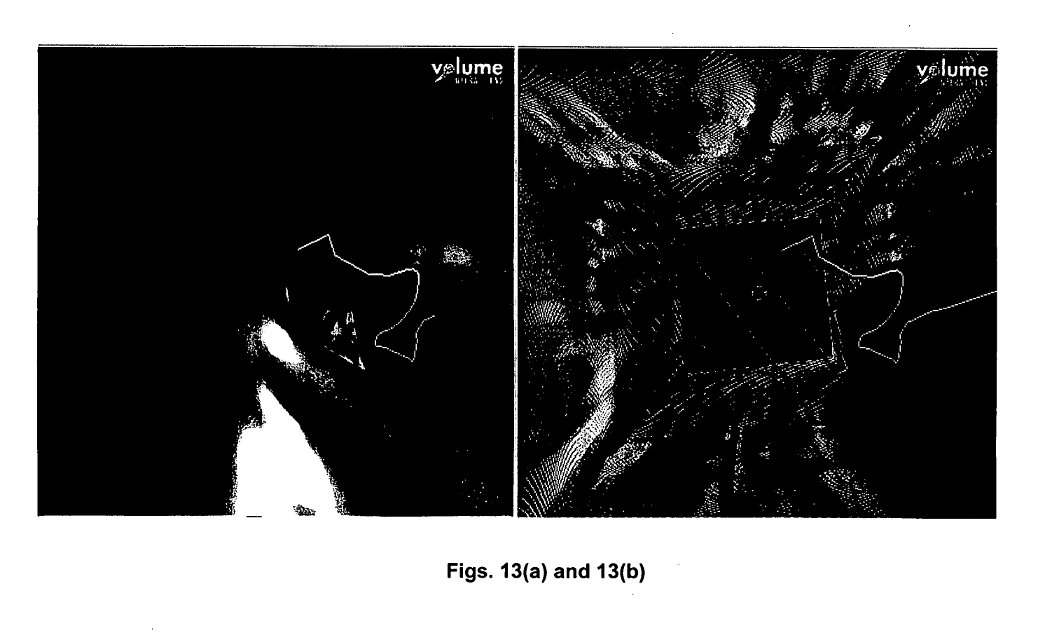

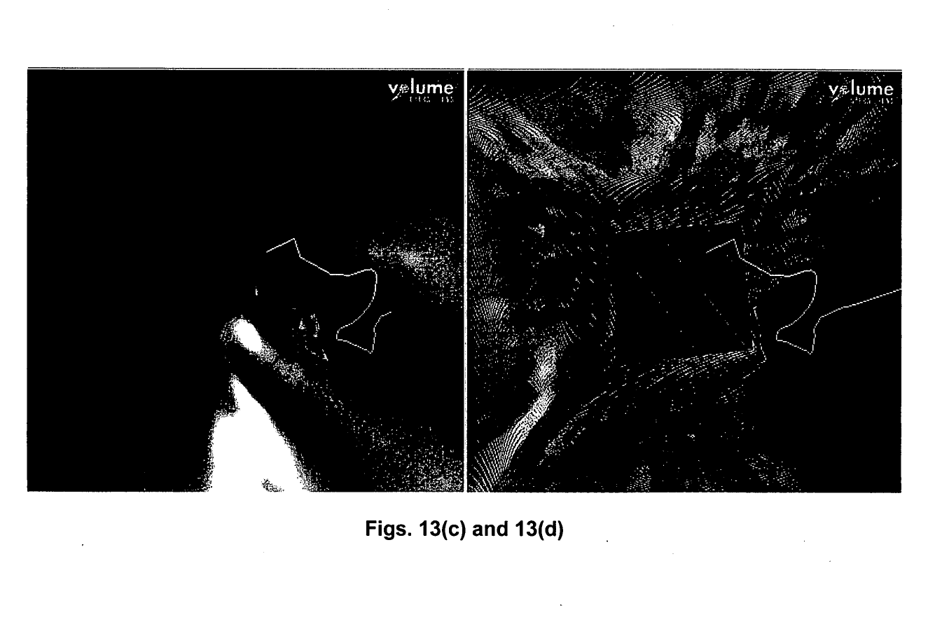

Finally, in FIGS. 13 the best image quality is seen, and these figures were generated using thousands of polygons. The edges of polygons are so close to each other that they appear to be connected into faces in the right part of the images (i.e., 13(b) and (d)).

One inelegant method of obtaining a crop box that can enclose all visible voxels is to shoot out a number of rays equal to the number of pixels used for the display, thus covering the entire screen area. However, if the screen area is, for example, 512 by 512 pixels, this requires shooting approximately 512×512=262,144 rays. Such a method is often impractical due to the number of pixels and rays involved which must be processed.

Thus, in exemplary embodiments of the present invention, a group of rays can be shot, whose resolution, for example, is sufficient to capture the shape of the visible boundary. This type of group of rays is shown in cyan (black crosses) in FIG. 3.

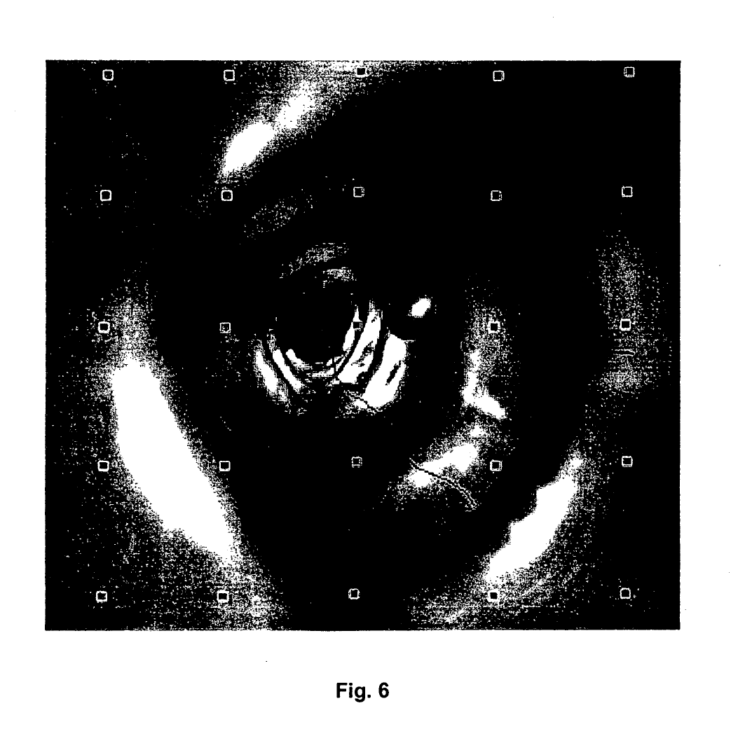

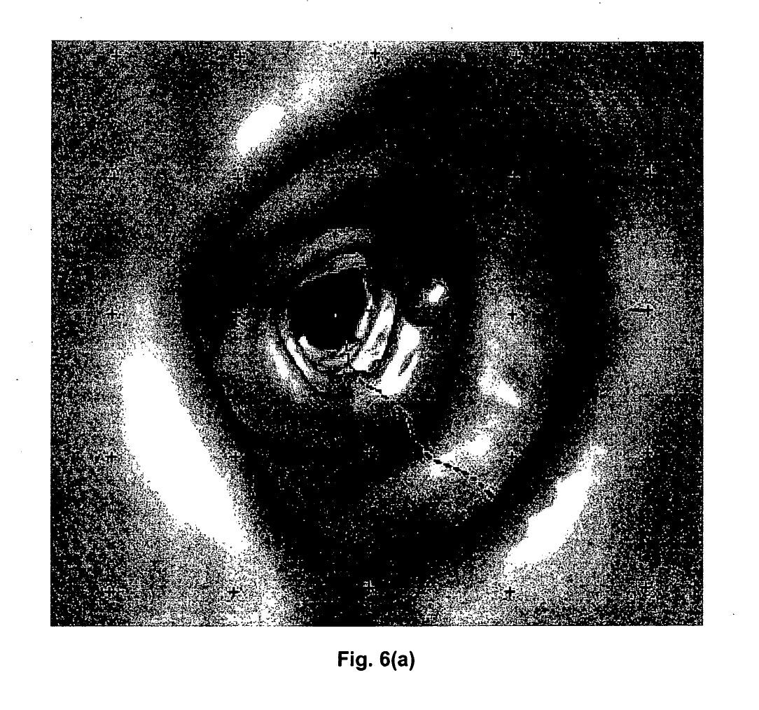

As can be seen from FIGS. 3 and 6, where an exemplary colon is depicted, often the greatest depth at a particular viewpoint is most pronounced at the rear of the centerline. This is because in an endoscopic view a user is generally looking into the colon, pointing either towards the cecum or towards the rectum. Thus, uniformly distributed rays (shown as cyan rays or black crosses in FIGS. 3 and 6) shot throughout the volume of the colon will not hit the farthest boundary of the visible voxels. If the distance between rays (what can be termed their “resolution”) is greater than (in this case) the diameter of the, for example, colon lumen at the back of the image then the shot rays may all return hit points too close to the viewpoint to include the back portion of the colon lumen in the crop box. Thus, in FIG. 6, the back part of the tube-like structure is not displayed and black pixels fill the void. To remedy this, in exemplary embodiments of the present invention, a centerline (or other area known to correlate with a portion of the visibility box missed by the first set of low resolution rays shot) may be examined in order to determine where the further end of the visible part of the “tube” is with respect to the screen area.

In exemplary embodiments of the present invention, this can be implemented, for example, as follows:

In previous pseudo code for distribute_rays:

After step 3,
4. determine the area of interest by finding out where the centerline leads to;
5. Further divide the part of the project plane containing this area of interest into smaller grids; and
6. Shoot one ray towards the center of each grid.

Step (4) can be implemented, for example, as follows. Since, in exemplary embodiments of the present invention, an exemplary program can have the position of the current viewpoint, as well as its position on the centerline and the shape of centerline, the program can, for example, simply incrementally check along the current direction to a point N cm away on the centerline, until such point is not visible any more; then on the projection plane, it can, for example, determine the corresponding position of the last visible point:

Exemplary Pseudocode To Determine Area Of Interest (Step 4 Above):
1. Get current viewpoint position P₀;
2. Get the relative position of current viewpoint position P₀ on the centerline (in terms of how many CMs away from the beginning of the centerline)
3. Get the centerline point Pᵢ that is (n×i) centimeter away from current view point (say n=5 cm);
4. Check if P₀ and Pᵢ are visible to each other, by shooting a ray from P₀ to Pᵢ:
   If there exists a hit point between P₀ and Pᵢ (which means the ray hit before it reaches Pᵢ), then P₀ and Pᵢ are invisible; return P₍ᵢ₋₁₎;
   Else: i=i+1; go to 3;

Step (5) can be implemented, for example, as follows:

Exemplary Pseudocode For Grid Subdivision (Step 5 Above):
For the last visible point calculated in previous step,
1. Get the projection of this point on the projection plane;
2. Take a rectangular area centered at this point on the projection plane, of size 1/m of the whole projection plane (in practice, for example, set m=5); and
3. divide this rectangular area into m by m grids (for m=5, 25 grids).

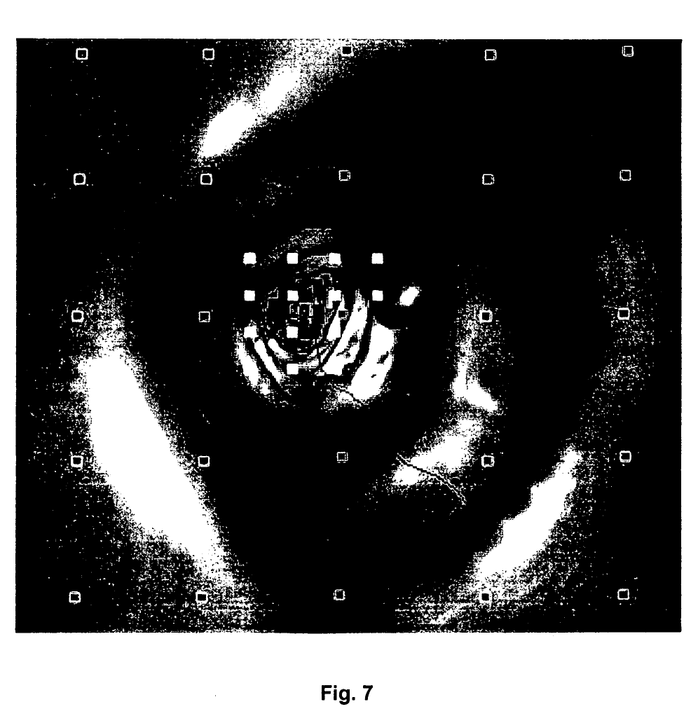

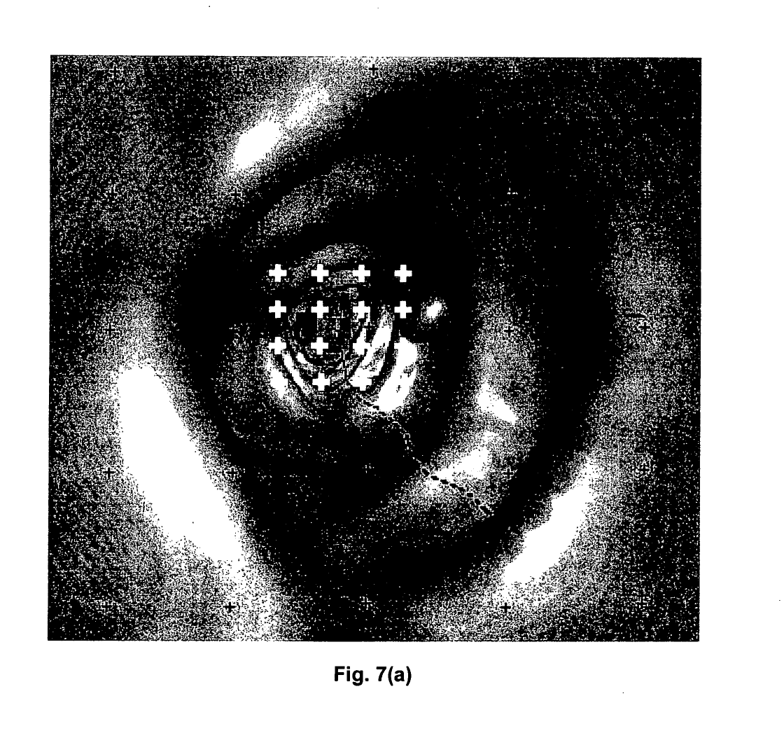

Thus, in exemplary embodiments of the present invention, a system can, for example, shoot additional rays centered at the end of the centerline in order to fill the missing part using the ray shooting method described above, but with a much greater resolution, or a much smaller spacing between rays. The result of this method is illustrated in FIG. 7, where the tube-like structure no longer has a missing part, as the second set of rays (shown in yellow or white crosses in FIG. 7) have obtained sufficient hit points along the actual boundary to capture its shape and thus adequately enclose it in a crop box.

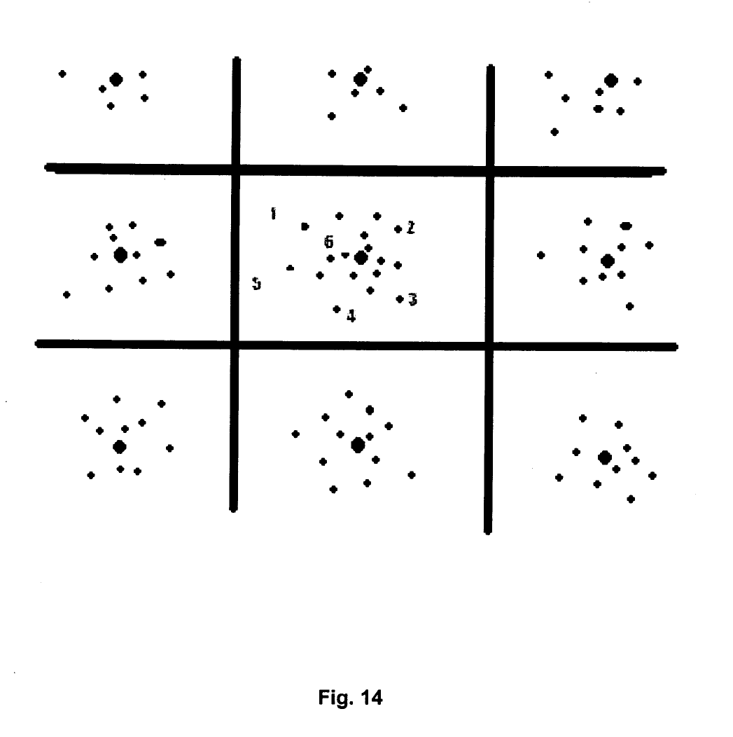

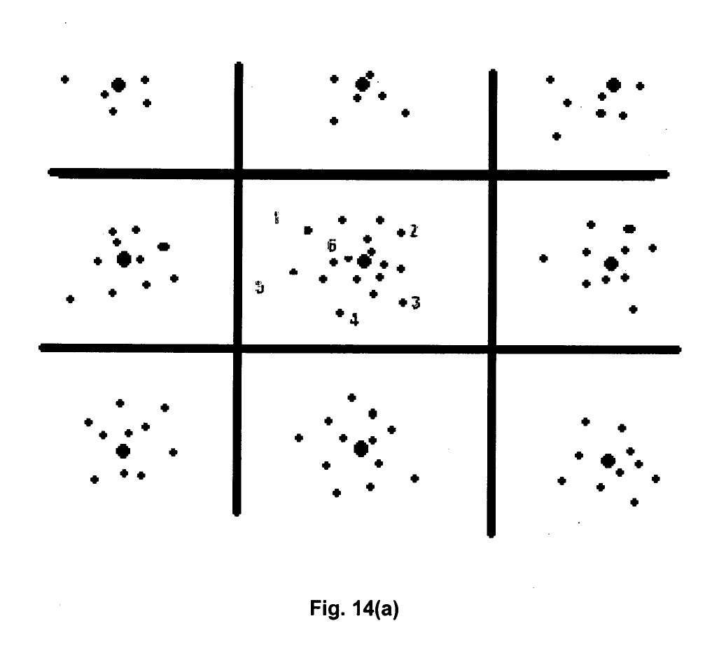

Given the situation depicted in FIG. 6, in alternate exemplary embodiments of the present invention, it may not be useful to constantly shoot rays in fixed directions, when trying to better capture the dimensions of a required crop box. Rather, in such embodiments, ray shooting can be performed, for example, using a random offset, so that the distance between hit points is not uniform. This can obviate the “low resolution” of shot rays problem described above. Such a technique is illustrated in FIG. 14, where in each loop the numbers 1, 2, . . . , 6 represent rays shot in each of loops 1, 2, . . . , 6 respectively, each time with a different, randomized offset.

Thus, with reference to FIG. 14, using the exemplary pseudocode for distribute_rays as provided above, an exemplary implementation could, for example, not just shoot one ray towards the exact center of each grid, but could, for example, randomize each ray's direction, such that the ray's direction (dx, dy) becomes (dx+random_offset, dy+random_offset).

Using such an exemplary technique, the total number of rays shot remains the same, but rays in consecutive frames are not sent along identical paths. This method can thus, for example, cover the displayed area more thoroughly than using a fixed direction of rays approach, and can, in exemplary embodiments, obviate the need for a second set of more focused (“higher resolution”) rays, such as are shown in FIG. 7, that are shot into a portion of the volume where the boundary is known to have a small aperture (relative to the inter-ray distance of the first set of rays) but with large +Z co-ordinates (i.e., it extends a far distance into the screen away from the viewpoint).

Exemplary Systems

The present invention can be implemented in software run on a data processor, in hardware in one or more dedicated chips, or in any combination of the above. Exemplary systems can include, for example, a stereoscopic display, a data processor, one or more interfaces to which are mapped interactive display control commands and functionalities, one or more memories or storage devices, and graphics processors and associated systems. For example, the Dextroscope™ and Dextrobeam™ systems manufactured by Volume Interactions Pte Ltd of Singapore, running the RadioDexter software, or any similar or functionally equivalent 3D data set interactive display systems, are systems on which the methods of the present invention can easily be implemented.

Exemplary embodiments of the present invention can be implemented as a modular software program of instructions which may be executed by an appropriate data processor, as is or may be known in the art, to implement a preferred exemplary embodiment of the present invention. The exemplary software program may be stored, for example, on a hard drive, flash memory, memory stick, optical storage medium, or other data storage devices as are known or may be known in the art. When such a program is accessed by the CPU of an appropriate data processor and run, it can perform, in exemplary embodiments of the present invention, methods as described above of displaying a 3D computer model or models of a tube-like structure in a 3D data display system.

While this invention has been described with reference to one or more exemplary embodiments thereof, it is not to be limited thereto and the appended claims are intended to be construed to encompass not only the specific forms and variants of the invention shown, but to further encompass such as may be devised by those skilled in the art without departing from the true scope of the invention.

1. A method for optimizing the dynamic displaying of a 3D data set, comprising:
determining the visible boundaries of a relevant portion of a 3D data set from a current viewpoint;
displaying said relevant portion of the 3D data set; and
repeating said determining and said displaying processes each time the co-ordinates of the current viewpoint change.

2. The method of claim 1, wherein the relevant portion of the 3D data set is an endoscopic view of a tube-like structure.

3. The method of claim 1, wherein said determining the visible boundaries is implemented by shooting rays from the current viewpoint to the surrounding inner walls of the tube-like structure and obtaining a set of hit points.

4. The method of claim 1, wherein the relevant portion of the 3D data set is an endoscopic view of a colon.

5. The method of claim 4, wherein said determining the visible boundaries is implemented by shooting rays from a viewpoint on a colon lumen centerline to the surrounding inner walls of the colon and obtaining a set of hit points.

6. The method of claim 3, wherein said rays are shot from a viewpoint on a centerline of the tube-like structure and are distributed so as to cover a visible area. from the viewpoint

7. The method of claim 6, wherein said rays are evenly distributed over said visible area.

8. The method of claim 6, wherein the direction in which said rays are shot includes a random component.

9. The method of claim 3, wherein:
each point in the data set can be characterized by co-ordinates along each of three axes A, B and C;
the minimum and maximum values for each co-ordinate over the set of hit points are found as Amin, Amax, Bmn, Bmax, Cmin, Cmax; and
the visible boundaries from the viewpoint comprise a closed surface having one corner at (Amin, Bmin, Cmin) and an opposite corner at (Amax,Bmax,Cmax).

10. The method of claim 9, wherein A, B and C are orthogonal directions in Euclidean space.

11. The method of claim 9, wherein A, B and C are components of a polar co-ordinate system.

12. The method of claim 9 wherein A, B, and C are either parallel or equal to the axes used internally by a system displaying the 3D data set to designate the 3D data set.

13. The method of claim 9, wherein A, B, and C are aligned with the viewing frustum of the current viewpoint.

14. The method of claim 3, wherein:
a first set of rays are shot into a first area from the current viewpoint within the tube-like structure at a first resolution; and
a second set of rays are shot from the current viewpoint towards a second area at a second resolution,
wherein the second area is a subset of the first area.

15. The method of claim 14, wherein the second area is determined to be possibly inadequately sampled by the first set of rays.

16. The method of claim 14, wherein the defined area is determined by checking an area of the tube-like structure surrounding a direction where visible voxels with greatest distance from viewpoint are found.

17. The method of claim 14, wherein the defined area is determined by checking where the centerline becomes invisible in the current scene.

18. The method of claim 3, wherein at each point along a centerline within a tube-like structure where rays are shot, said rays are shot from each of two viewpoints representing the positions of human eyes.

19. The method of claim 9, wherein at each point along a centerline within a tube-like structure where rays are shot, said rays are shot from each of two viewpoints representing the positions of human eyes.

20. A computer program product comprising a computer usable medium having computer readable program code means embodied therein, the computer readable program code means in said computer program product comprising means for causing a computer to:
determine the boundaries of a relevant portion of a 3D data set from a current viewpoint;
display said relevant portion of the 3D data set; and
repeat said determining and said displaying processes each time the co-ordinates of the current viewpoint change.

21. A program storage device readable by a machine, tangibly embodying a program of instructions executable by the machine to perform a method for optimizing the dynamic display of a 3D data set, said method comprising:
determining the boundaries of a relevant portion of a 3D data set from a current viewpoint;
displaying said relevant portion of the 3D data set; and
repeating said determining and said displaying processes each time the co-ordinates of the current viewpoint change.

22. The computer program product of claim 14, wherein said means further cause a computer to:
shoot a first set of rays into a first area from a current viewpoint within the tube-like structure at a first resolution; and
shoot a second set of rays from the current viewpoint towards a second area at a second resolution,
wherein the second area is a subset of the first area and s determined to be possibly inadequately sampled by the first set of rays.

23. The program storage device of claim 15, wherein said method further comprises:
shooting a first set of rays into a first area from a current viewpoint within the tube-like structure at a first resolution; and
shooting a second set of rays from the current viewpoint towards a second area at a second resolution,
wherein the second area is a subset of the first area and is determined to be possibly inadequately sampled by the first set of rays.
